(19) United States
(12) Patent Application Publication
(10) Pub. No.: US 2010/0030245 A1
(43) Pub. Date: Feb. 4, 2010

(54) MAGNETIC DEVICES FOR HIATAL HERNIA REMODELING

(76) Inventors: Ghassan S. Kassab, Zionville, IN (US); Jose A. Navia, Buenos Aires (AR)

Correspondence Address: ICE MILLER LLP, ONE AMERICAN SQUARE, SUITE 3100, INDIANAPOLIS, IN, 46282-0200, US

(21) Appl. No.: 12/307,113

(22) PCT Filed: Jun. 29, 2007
(86) PCT No.: PCT/US2007/015267

§ 371 Date: Aug. 27, 2009

Related U.S. Application Data
(60) Provisional application No. 60/817,423, filed on Jun. 30, 2006.

(51) Int. Cl. A61B 17/08

(52) U.S. Cl. 606/157

(57) ABSTRACT

Devices and methods are disclosed for providing static and dynamic tissue and organ restriction. A magnetic component is used to stabilize an apparatus to provide tissue and organ restriction. Such a device is described with respect to occluding a hernia via a mesh attachment, and, alternatively, with respect to closing or reinforcing the crural closure via a parallel magnetic arrangement.

### RELATED APPLICATIONS

This U.S. Utility patent application claims priority to U.S. Provisional Patent Application Ser. No. 60/817,423, filed Jun. 30, 2006.

### FIELD

The field relates generally to clamps, and more particularly, to magnetic tissue clamps used to occlude openings in tissue.

### BACKGROUND

The diaphragm is a sheet of muscle separating the abdomen and the chest cavity. The oesophagus traverses through the chest, passing through a small opening in the diaphragm called a hiatus, and enters the abdominal cavity proximate to the stomach. The oesophageal hiatus is situated in the muscular part of the diaphragm at the level of the tenth thoracic vertebra, and is elliptical in shape. The oesophageal hiatus is positioned above, in front, and slightly to the left of the aortic hiatus, and transmits the oesophagus, the vagus nerves, and some small oesophageal arteries. The right crus of the diaphragm loops around in such a manner so as to form a sling around the diaphragm. Upon inspiration, this sling constricts the diaphragm, forming an anatomical sphincter that prevents stomach contents from refluxing up the oesophagus when intra-abdominal pressure rises during inspiration.

However, when a hiatus hernia occurs, a portion of the stomach slides upwards through the hiatus in the diaphragm, adjacent to the oesophagus, and into the chest. Due to the positioning of the stomach, the sling is unable to constrict the diaphragm around the oesophagus to form a functional sphincter. Accordingly, when the intra-abdominal pressure rises, due to obesity or numerous other factors, there is nothing to prevent the stomach contents from refluxing up the oesophagus and a number of complications typically result.

Gastro-oesophageal reflux disease (“GERD”) includes all of the clinical manifestations when the oesophagus is exposed to the gastric content. In the United States, $1 billion dollars is spent annually on oesophageal diseases. Patients with chronic GERD are at an increased risk of oesophageal adenocarcinoma. Thus, a significant connection exists between obesity and oesophageal adenocarcinoma in the United States. (Barak et al., 2002).

As previously noted, the lower oesophageal sphincter (“LES”) and the crural portion of the diaphragm function as an anti-reflux barrier. The majority of patients with complicated GERD have a hiatal hernia that disrupts this anti-reflux barrier. Accordingly, a hiatal hernia induces GERD by: 1) deteriorating acid clearance from the oesophagus after swallowing; 2) reducing the crural diaphragm's function as a sphincter, and 3) eliminating the oesophagus' intra-abdominal portion's flap-valve mechanism which typically functions as a barrier to oesophageal exposure to gastric acid. (Barak et al., 2002).

In the last ten years, the laparoscopic technique has revolutionized the approach to hiatal hernia repair and the treatment of GERD. Laparoscopic surgical treatment of large diaphragmatic hiatal hernia is more popular than the traditional surgical treatments, such as thoracotomy or laparotomy approaches. Even when laparoscopic techniques are used, the surgical reduction of a hernia typically requires wide dissection, mobilization of the oesophagus and stomach, and the hernia sac must be totally resected from the mediastinum. In addition, to prevent anatomical recurrence of the hernia and GERD, it is necessary to perform crural approximation and fundoplication as part of the surgical treatment. (Dahlberg et al., 2001).

Even when surgical procedures are a success, the hiatal repair often subsequently fails due to tissue tension. Recurrence of the hernia can be directly associated to the mean diameter of the hiatus (>10 cm in some cases). The anatomy of the diaphragmatic pillars is another important factor. The hiatal crus is a fleshy structure lacking tendinous reinforcement and the use of ordinary sutures may cut the muscle. If the hiatus is predominantly wide and the diaphragmatic pillars are necessarily approached with suturing, the lateral portions of the diaphragm close to the crura become tense, with probable risk of disruption. (Targarona et al., 2004).

Two laparoscopic surgical techniques conventionally exist for the repair of a hiatus hernia: Tension-Free Techniques and Non-Tension-Free Techniques (referring to the resulting tension—or lack thereof—of the lateral portions of the diaphragm after the procedure). Today, typically all hernia repair surgeries are Tension-Free. Nevertheless, performing a Tension-Free repair technique in the hiatus is extremely difficult due to the oblique situation of the pillars and the challenge of fixing the mesh. Moreover, the hiatus is an anatomically complex structure because the esophagus moves during respiratory excursion of the diaphragm. (Targarona et al., 2004).

In one example of a Tension-Free Technique, Paul et al. (Paul et al., 1997) proposed the use of a triangular or semilunar polytef patch positioned to occlude the anterior segment of the hiatus, which is fixed to the diaphragm with staples or stitches. In conjunction, the stomach is fixed to the abdomen and a fundoplication is performed. The same technique is used for the posterior segment of the hiatus. Alternatively, Basso et al. (Basso et al., 2000) recommended the placement of a piece of mesh just covering the defect below the diaphragm, overlapping both pillars laterally.

In Non-Tension-Free. Techniques, the most common method for hiatal closure is the use of simple stitches or a continuous suture to approach the crural. Teflon or Dacron pledgets or a polypropylene strip are conventionally used to avoid the cutting stitches effect. The pillar closure is covered by a long strip of mesh, which is positioned below the diaphragm in order to reduce the risk of dysphagia or erosion by avoiding the encircling of the oesophagus. (Targarona et al., 2004).

Currently, nonreabsorbable materials of biological origin or synthetic materials are also used to repair the hiatus hernia (e.g., PTFE, bovine pericardium, etc.) due to the softness of the materials, as well as the materials' reduced capacity to induce adhesions. The surfaces of such materials further avoid tight adhesion to the visceral face of the mesh, and the soft texture of the free margin in close contact with the esophagus presents a low risk of damaging the esophagus. (Targarona et al., 2004).

In laparoscopic hiatal hernia repair, it is conventionally recommended to use mesh reinforcement to avoid recurrent herniation. (Frantzides et al., 2002). However, despite the beneficial properties of the mesh materials, mesh positioned in the hiatus may provoke complications depending on the type of mesh or the device used for the fixation thereof. For example, complications may arise due to local fibrosis (dysphagia) or digestive lumen erosion. In addition, other complications may be influenced by the mechanism used to fix the mesh, particularly when staples or tackers are applied, and can injure the vital structures surrounding the hiatus. Teflon pledgets, in particular, may also corrode the fundus or induce fibrous retraction and dysphagia. (Targarona et al., 2004). In addition, there is a 42% recurrence rate even when pledgeted sutures are used in the presence of increased tension during a simple cruroplasty.

Articles discussing hiatal hernia procedures include:

Barak et al. Gastro-oesophageal reflux disease in obesity: pathophysiological and therapeutic considerations. Obes Rev 2002; 3(1): 9-15.

Basso N et al. 360 degrees laparoscopic fundoplication with tension-free hiatoplasty in the treatment of symptomatic gastroesophageal reflux disease. Surg Endosc 2000; 14(2): 164-69.

Dahlberg P S et al. Laparoscopic repair of large paraesophageal hiatal hernia. Ann Thorac Surg 2001; 72(4): 1125-29.

Frantzides C T et al. A prospective, randomized trail of laparoscopic polytetrafluoroethylene (PTFE) patch repair vs. simple cruroplasty for large hiatal hernia. Arch Surg 2002; 137(6): 649-52.

Paul M G et al. Laparoscopic tension-free repair of large paraesophageal hernias. Surg Endosc 1997; 11(3): 303-7.

Targarona E M et al. Mesh in the hiatus: a controversial issue. Arch Surg 2004; 139(12): 1286-96.

### SUMMARY

A magnetic apparatus is provided for occluding an opening in a tissue. The magnetic apparatus disclosed herein may be used to treat herniation. One embodiment of the apparatus includes at least one magnetic component that is biased to decrease the size of an opening in a tissue. In one embodiment, the at least one magnetic component may include a first magnetic component and a second magnetic component. These components can each be configured as a bar, or any other shape that is capable of occluding an opening in a tissue. In this embodiment, when the first magnetic component and the second magnetic component are in proximity to each other, the two components are biased together and magnetically engage, thereby compressing any tissue situated therebetween and securing the magnetic apparatus in place.

In an alternative embodiment, an apparatus is provided comprising a first component and a second component. The first component comprises a magnetic frame shape defining an interior and a section of mesh disposed across a portion of the interior. The second component comprises a magnetic frame shape that matches at least a portion of the magnetic frame shape of the first component. When the first component and the second component are in proximity to each other, the components are biased towards each other to thereby compress any tissue disposed therebetween (i.e. the sandwich effect). In one embodiment, this apparatus may be applied to treat a hiatal hernia, whereby the mesh occludes the hiatal opening to prevent the stomach from traversing therethrough.

A method is also provided for occluding a tissue opening. The method comprises the steps of providing an apparatus comprising a first magnetic component and a second magnetic component that are capable of magnetically engaging through a tissue. The method further comprises the step of adhering the apparatus to a tissue such that at least a portion of the opening of the tissue is occluded.

In yet another embodiment, an alternative method is provided for occluding a tissue opening. The method comprises the steps of providing a magnetic apparatus for placement on a tissue having an opening with a first circumference, the magnetic apparatus biased to decrease the size of the opening in the tissue, and placing the magnetic apparatus around the tissue to result in the opening having a second, smaller circumference.

An additional embodiment of the method comprises the steps of providing an apparatus comprising a first component having a first magnetic shape that defines an interior and a section of mesh disposed across a portion of the interior, and a second component having a second magnetic shape that matches at least a portion of the first magnetic shape of the first component. The method further comprises the step of positioning the apparatus such that a tissue having an opening is located between the first and second components, and allowing the first component and the second component to magnetically engage each other, thereby compressing the tissue therebetween and at least partially occluding the opening.

### BRIEF DESCRIPTION OF THE DRAWINGS

FIG. 1 shows a side view of one embodiment of the occlusion device for reducing the size of an opening.

FIG. 2 shows the occlusion device shown in FIG. 1 positioned proximate to the site of a hiatal hernia.

FIGS. 3A and 3B show graphical representations of the magnetostatic forces involved in the calculation of the field strength between the two parallel plates of the occlusion device of FIGS. 1 and 2.

FIG. 4 shows a flow chart of a method for using the occlusion device of FIGS. 1 and 2 to occlude an opening in a tissue.

FIG. 5 shows a schematic view of one embodiment of an occlusion device for occluding an opening in a tissue.

FIG. 6 shows a bottom view of one embodiment of the occlusion device of FIG. 5 positioned around the site of a hiatal hernia in an anterior placement.

FIG. 7 shows the embodiment of the occlusion device shown in FIG. 5 positioned around the site of a hiatal hernia in a posterior placement.

FIG. 8 shows a flow chart of a method for using the occlusion device of FIG. 5 to cover an opening in a tissue.

### DETAILED DESCRIPTION

Reference will now be made to the embodiments illustrated in the drawings and specific language will be used to describe the same. It will nevertheless be understood that no limitation of scope is intended by the description of these embodiments.

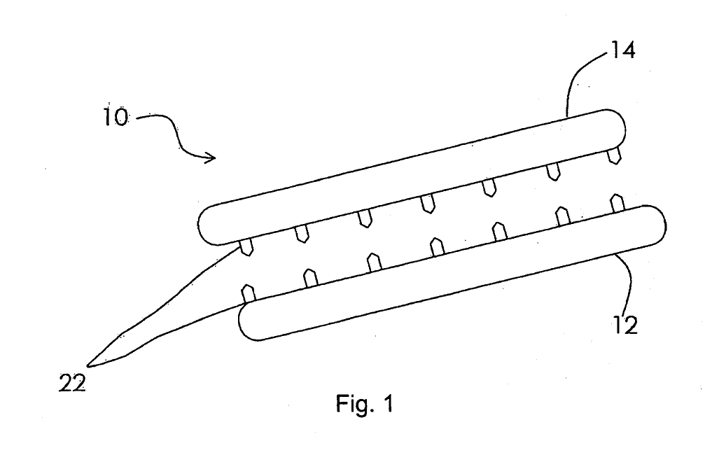

FIG. 1 shows a schematic view of one embodiment of an occlusion device 10 for reducing the size of an opening. In this embodiment, the occlusion device 10 is comprised of a first magnetic component 12 and a second magnetic component 14. The first magnetic component 12 is comprised of a first shape and the second magnetic component 14 comprises a second shape that matches at least a portion of the first shape of the first magnetic component 12. The first magnetic component 12 and the second magnetic component 14 are each comprised of any ferromagnetic material known in the art so long as the material is capable of magnetically engaging through a tissue. In addition, the first and second magnetic components 12, 14 may be flexible, semi-flexible, or articulated.

In the embodiment shown in FIG. 1, the first and second magnetic components 12, 14 each comprise a straight bar. It will be recognized that while the first and second magnetic components 12, 14 of FIG. 1 are shown as straight bars, any configuration may be used so long as the first and second components 12, 14 are capable of being inserted into a body cavity laparoscopically and are capable of magnetically engaging with each other through a tissue (i.e. the sandwich effect).

The first magnetic component 12 and the second magnetic component 14 are polarized such that the first and second magnetic components 12, 14 are biased towards each other. Due to the configuration of the second magnetic component 14 and the bias between the first magnetic component 12 and the second magnetic component 14, the first and second magnetic components 12, 14 are capable of magnetically engaging. When the first and second magnetic components 12, 14 magnetically engage, the two components form a single unit that is secured to any tissue disposed between the two magnetic components 12, 14.

In one embodiment, the occlusion device 10 may further comprise a plurality of barbs 22. In this embodiment, the barbs 22 extend from both the first magnetic component 12 and the second magnetic component 14 such that the barbs 22 mechanically engage the opposite magnetic component when the first and second magnetic components 12, 14 are in close proximity and magnetically engaged. In this manner, the plurality of barbs 22 function to reinforce the engagement between the first magnetic component 12 and the second magnetic component 14 when the first and second magnetic components 12, 14 are in close proximity.

The plurality of barbs 22 may be sharp, pointed, or dull and comprise any material known in the art that does not interfere with the magnetic engagement between the first magnetic component 12 and the second magnetic component 14. In an additional embodiment, each of the barbs 22 protruding from the first and second magnetic components 12, 14 has a corresponding indentation (not shown) located on the opposite magnetic component. Accordingly, when the first magnetic component 12 and the second magnetic component 14 mechanically engage, each of the plurality of barbs 22 is received by its corresponding indentation in the opposite magnetic component.

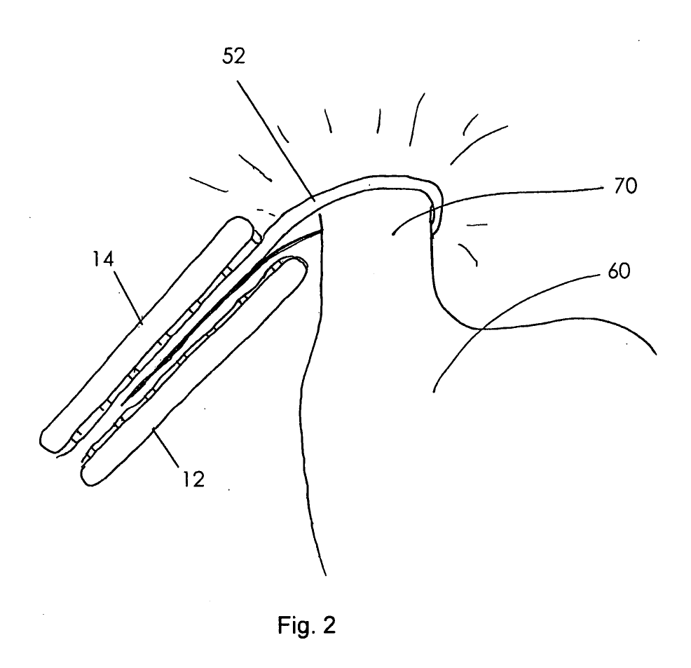

FIG. 2 shows a bottom view of the occlusion device 10 implanted for use to treat and/or prevent a hiatal hernia. In this embodiment, the occlusion device 10 is employed as a “Non-Tension Free” device because the placement of the occlusion device 10 relative to the diaphragm 52 creates tension on the crus of the diaphragm 52. As previously discussed, the esophagus 70 passes through a “hiatus”, or opening, in the diaphragm wall 52 before reaching the stomach 60. As a consequence of either physical debilities attending acid reflux disease, obesity, or other medical ailments, the esophageal hiatus may become enlarged and a hiatal hernia may develop. A hiatal hernia is a protrusion of the stomach upward into the mediastinal cavity through the esophageal hiatus of the diaphragm 52. By applying the occlusion device 10 to the diaphragm 52 adjacent to the esophageal hiatus, the condition can be corrected and/or avoided altogether.

In FIG. 2, the occlusion device 10 is shown coupled with the diaphragmatic wall 52 in a location adjacent to both the stomach 60 and the esophagus 70. In this embodiment, the first and second magnetic components 12, 14 each comprise a straight bar configuration. In application, both the first magnetic component 12 and the second magnetic component 14 are positioned adjacent to the inferior wall of the diaphragm 52 or, alternatively, the superior wall of the diaphragm 52. As shown in FIG. 2, the occlusion device 10 is positioned adjacent to the inferior wall of the diaphragm 52 in an anterior placement.

When the first magnetic component 12 and the second magnetic component 14 are positioned in close proximity to one another, the first and second magnetic components 12, 14 are biased towards each other. To treat and/or prevent a hiatal hernia, the first and second magnetic components 12, 14 are positioned adjacent to the edges of the enlarged esophageal hiatus and allowed to magnetically engage one another such that a portion of the diaphragm wall 52 is disposed and compressed therebetween. The compression resulting from the magnetic engagement of the occlusion device 10 compresses the edges of the esophageal hiatus together and thereby reduces the size of the diaphragmatic opening. Depending on the specific placement of the occlusion device 10, the occlusion device 10 may be operable to reduce the size of the esophageal hiatus so that the hiatus is only approximately the diameter of the esophagus 70 traversing therethrough. By removing the additional space from the hiatus, the placement and structure of the occlusion device 10 prevent the stomach 60 from protruding through the diaphragm wall 52 and into the mediastinal cavity via the esophageal hiatus.

In an alternative embodiment of the occlusion device 10, the occlusion device 10 may comprise a single magnetic component (not shown). In this embodiment, at least a portion of the magnetic component is flexible or semi-flexible, such that at least a section of the magnetic component is capable of folding. The magnetic component of this embodiment comprises a first end and a second end. In operation, the portion of the magnetic component that is flexible or semi-flexible folds, such that the first end and the second end can magnetically engage each other. In this manner, the single magnetic component can function as a clamp. For example, the magnetic component of this embodiment is operable to clamp a portion of the edge of an enlarged esophageal hiatus (in the manner illustrated in FIG. 2), thereby resulting in an esophageal hiatus with a smaller diameter. In addition, the magnetic component of this embodiment may comprise a plurality of barbs 22 and corresponding indentations. As this embodiment of the occlusion device 10 only comprises one magnetic component, the barbs 22 and the corresponding indentations are necessarily both located on the same component of the occlusion device 10.

The magnetic force between the first and second magnetic components 12, 14 of the occlusion device 10 reduces the risk that the occlusion device 10 will become displaced or dislodged over time. In addition, in the embodiments where the occlusion device 10 further comprises a plurality of barbs 22, the plurality of barbs 22 further secure the occlusion device 10 to the diaphragm wall 52, and/or the first magnetic component 12 to the second magnetic component 14, thereby further securing the occlusion device 10 to the diaphragm wall 52 and preventing the occlusion device 10 from shifting or becoming dislodged. For example, depending on the configuration of the barbs 22, the barbs 22 may form a wave-like pattern in the diaphragm tissue 52 and increase the amount of force required to dislodge the occlusion device 10 from the diaphragm 52. Alternatively, the barbs 22 may be configured as sharp points such that the barbs 22 puncture the diaphragmatic tissue 52 and thus function similarly to sutures or staples by securing the occlusion device 10 to the diaphragm 52. Accordingly, the barbs 22 can provide further resistance and prevent the occlusion device 10 from shifting relative to the esophagus 70 or the diaphragm 52.

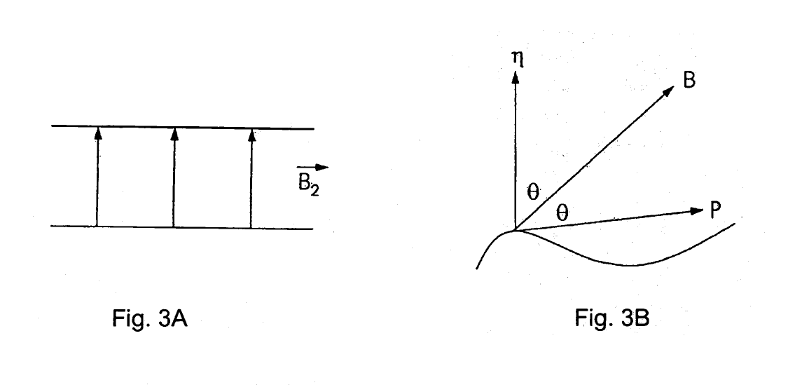

A user of the occlusion device 10 (e.g., a physician) may also select specific permanent magnets to comprise the first and second magnetic components 12, 14 such that the first and second magnetic components 12, 14 exert an optimal amount of magnetostatic force to promote the stabilization of the occlusion device 10. For the theoretical application of the occlusion device 10 in the stomach of obese persons, an example calculation is provided below. In light of the two parallel plates shown in FIG. 3A, the Maxwell's stress tensor is written as follows:

$${T}_{ij} = \frac{1}{μ} [ {B}_{i} {B}_{j} - \frac{1}{2} {B}^{2} {δ}_{ij} ] \qquad [ 1 ]$$

Since only B̅_{z} exists in this application, the Maxwell's stress tensor is written as:

$${T}_{ij} = [ \begin{matrix}- \frac{{\vert {B}_{z} \vert}^{2}}{2 μ} & 0 & 0 \\ 0 & - \frac{{\vert {B}_{z} \vert}^{2}}{2 μ} & 0 \\ 0 & 0 & \frac{{\vert {B}_{z} \vert}^{2}}{2 μ}\end{matrix} ] \qquad [ 2 ]$$

The stress tensor vector which is normal to the surface in two-dimensional coordinates has the form:

$$P = [ \begin{matrix}- \frac{{\vert {B}_{z} \vert}^{2}}{2 μ} & 0 & 0 \\ 0 & - \frac{{\vert {B}_{z} \vert}^{2}}{2 μ} & 0 \\ 0 & 0 & \frac{{\vert {B}_{z} \vert}^{2}}{2 μ}\end{matrix} ] ( \begin{matrix}0 \\ 0 \\ {n}_{z}\end{matrix} ) = \frac{{\vert {B}_{z} \vert}^{2}}{2 μ} \qquad [ 3 ]$$

where, if |B_{z}|=0.5 T, the pressure is calculated as follows:

$$P = \frac{{\vert {B}_{z} \vert}^{2}}{2 μ} = \frac{{0.5}^{2}}{8 π \times {10}^{- 7}} = 99.47 ( k Pa ) \qquad [ 4 ]$$

If it is assumed that the angle between the magnetic field B and normal direction of the magnetic plate is taken as 15°, and area=[2π×(1.0×10⁻²)]×(0.5×10⁻²) m² (illustrated in FIG. 3B), the force is calculated as follows:

F=P×sin 30°×area=99.47×0.5×π×0.1=15.62 (Newton) [5]

The force determined by Equation 5 represents the tangential force required to oppose or resist movement or migration of the occlusion device 10. Accordingly, the occlusion device 10 can be designed to yield a required force. The area of the occlusion device 10 may also be appropriately designed to spread out the force in order to minimize the compression of the tissue. Other forces may be similarly determined for different geometries and areas under consideration.

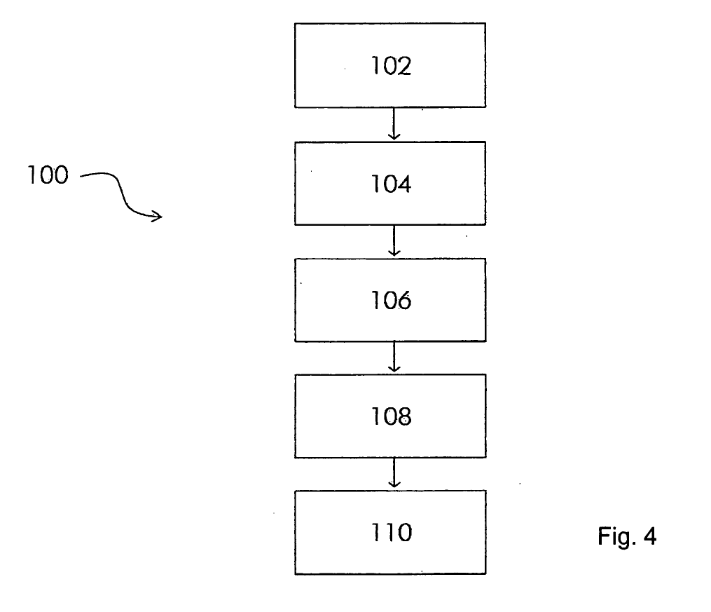

FIG. 4 shows a flow chart of a method 100 for reducing the size of an esophageal hiatus by employing the occlusion device 10. For ease of understanding, the steps of the related methods described herein will be discussed relative to the components of the occlusion device 10 shown in FIGS. 1 and 2, but it will be appreciated by one skilled in the art that any such device can be used to perform these methods, so long as the device is capable of magnetically engaging a magnetic composition through a piece of tissue, such that the engagement is secure.

At step 102, the first and second magnetic components 12, 14 are inserted laparoscopically into the patient's body. In this embodiment and the embodiment where the occlusion device 10 comprises a single flexible magnetic component, the magnetic component(s) may be inserted through a catheter into the patient's abdominal cavity. At step 104, the first and second magnetic components 12, 14 are positioned adjacent and parallel to the inferior surface of the diaphragm 52. Downward tension is applied to the edges of the enlarged portion of the esophageal hiatus at step 106 and the edges are folded down into the abdominal cavity. In this manner, the superior surfaces of the diaphragm 52 on each side of the esophageal hiatus are positioned adjacent to each other and are in physical communication. In an alternative embodiment of the method 100, the occlusion device is positioned adjacent and parallel to the superior surface of the diaphragm 52 at step 104. In this embodiment, at step 106 upward tension is applied tot eh edges of the enlarged portion of the esophageal hiatus. Accordingly, the edges are folded up into the thoracic cavity.

In both embodiments of the method 100, at step 108 the first magnetic component 12 is positioned on one side of the pinched edges of the hiatus, and the second magnetic component 14 is positioned on the opposite side of the pinched edges of the hiatus. The first and second magnetic components 12, 14 are then allowed to magnetically engage at step 110, such that the edges of the hiatus are compressed therebetween. In securing the edges of the hiatus in such a manner, the occlusion device 10 decreases the size of, thereby occludes, the esophageal opening.

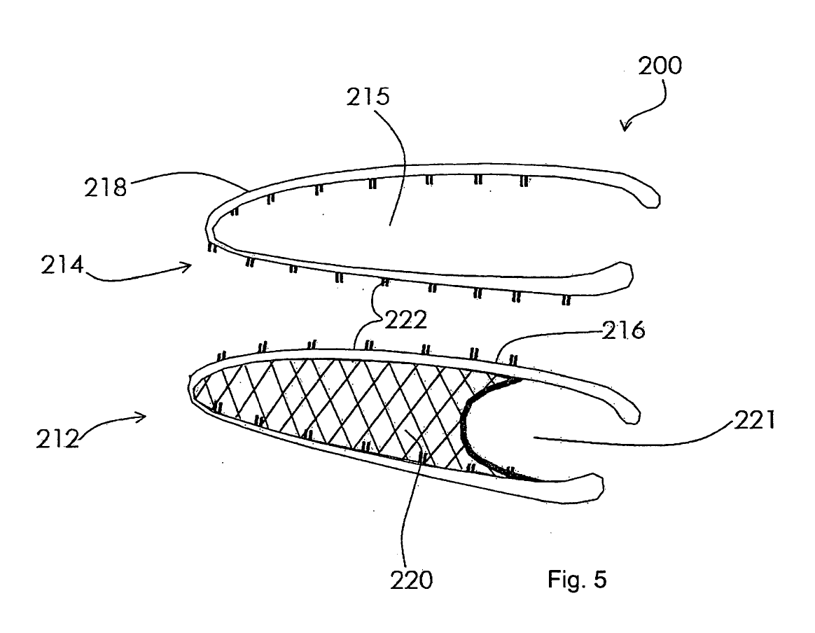

FIG. 5 shows a schematic view of an alternative embodiment of the occlusion device 10. The only difference between the occlusion device 10 and the occlusion device 200 is that the magnetic components of the occlusion device 200 may each define an interior. In the embodiment shown in FIG. 5, the occlusion device 200 is comprised of a first magnetic component 212 and a second magnetic component 214. The first magnetic component 212 comprises a first magnetic shape 216 that defines an interior area 215. The first magnetic component 212 may be comprised of any ferromagnet known in the art that is capable of magnetically engaging the second magnetic component 214 through a tissue and may be flexible, semi-flexible, or articulated.

The first magnetic component 212 further has a section of mesh 220 disposed across a portion of the interior 215 defined by the first magnetic shape 216. The mesh 220 may be comprised of any non-reabsorbable material, whether synthetic or biological, that is known in the art. Examples of such non-reabsorbable materials include, but are not limited to, polytetrafluoroethylene, polyurethane, or pericardium. Because the mesh 220 is not disposed across the totality of the interior 215 of the first magnetic component 212, an opening 221 is defined. The opening 221 may comprise any size or configuration necessary for the desired application of the occlusion device 200. The first magnetic shape 216 may be configured in any shape so long as the first magnetic shape 216 defines the interior 215 and at least one end of the first magnetic shape 216 is open. Merely by way of example, and without any intended limitation, the first magnetic shape 216 may be configured as in a rectangular shape, C-shape, U-shape, or V-shape.

The second magnetic component 214 comprises a second magnetic shape 218 that matches at least a portion of the first magnetic shape 216 of the first magnetic component 212. As shown in FIG. 5, the second magnetic shape 218 may comprise a U-shape. While the embodiment of the second magnetic shape 218 shown in FIG. 5 defines the interior area 215, it will be recognized that the second magnetic shape 218 of the second magnetic component 214 need not define the interior area 215 so long as the second magnetic shape 218 matches at least a portion of the first magnetic shape 216. The second magnetic component 214 may be comprised of any ferromagnet known in the art that is capable of magnetically engaging the first magnetic component 212 through a tissue. In addition, the second magnetic component 214 may be flexible, semi-flexible, or articulated.

In the embodiment shown in FIG. 5, the first magnetic component 212 and the second magnetic component 214 comprise permanent magnets having U-shape configurations. The first magnetic component 212 comprises the mesh 220 that extends radially inward from the first magnetic shape 216. In this embodiment, the opening 221 is disposed proximate to the open end of the first magnetic component 212. In an alternative embodiment, the first magnetic component 212 and the second magnetic component 214 both comprise the mesh 220 extending across the interior 215 such that a double layer of mesh 220 is provided when the first and second magnetic components 212, 213 magnetically engage.

The first magnetic component 212 and the second magnetic component 214 are polarized such that they are biased towards each another. Due to the matching configuration and the bias between the first magnetic component 212 and the second magnetic component 214, the first and second magnetic components 212, 214 are capable of magnetically engaging. When the first and second magnetic components 212, 214 magnetically engage, a single unit is formed and secured to any tissue disposed between the two magnetic components 212, 214.

Similar to the occlusion device 10, in one embodiment of the occlusion device 200, the first and second magnetic components 212, 214 further comprise a plurality of barbs 222. In this embodiment, the plurality of barbs 222 extend from both the first and second magnetic components 212, 214 such that the barbs 222 mechanically engage the opposite magnetic component when the first magnetic component 212 and the second magnetic component 214 are in close proximity and magnetically engaged. In this manner, the plurality of barbs 222 function to reinforce the engagement between the first magnetic component 212 and the second magnetic component 214.

Similar to the barbs 22 described in conjunction with one embodiment of the occlusion device 10, the barbs 222 may be sharp, pointed, or dull and comprise any material known in the art that does not interfere with the magnetic engagement between the first magnetic component 212 and the second magnetic component 214. In one embodiment, each of the barbs 222 protruding from the first and second magnetic components 212, 214 has a corresponding indentation (not shown) located on the opposite magnetic component. Accordingly, when the first magnetic component 212 and the second magnetic component 214 mechanically engage, each of the plurality of barbs 222 is received by its corresponding indention in the opposite magnetic component. In this manner, the plurality of barbs 222 and the corresponding indentations function to secure the first magnetic component 212 to the second magnetic component 214.

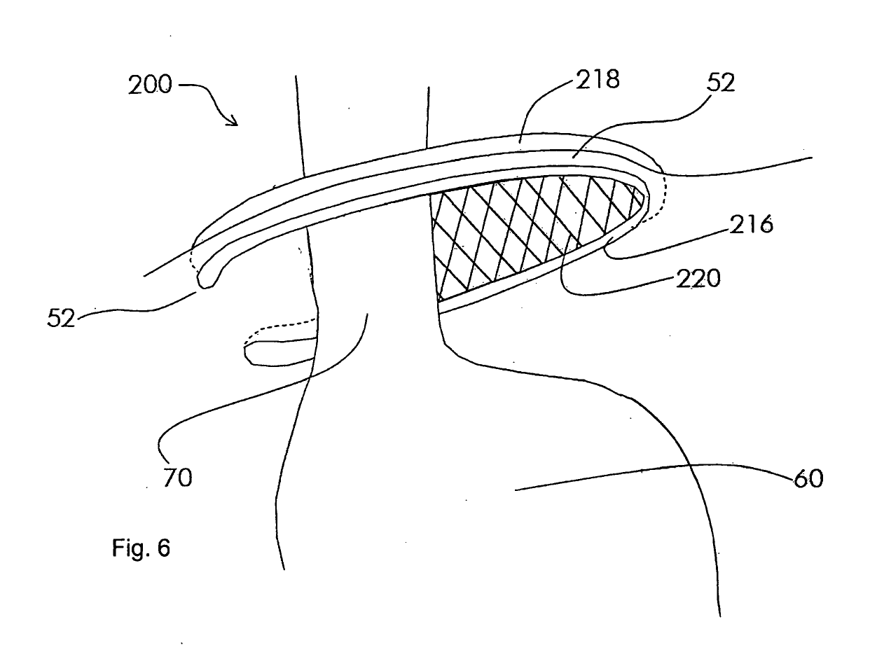

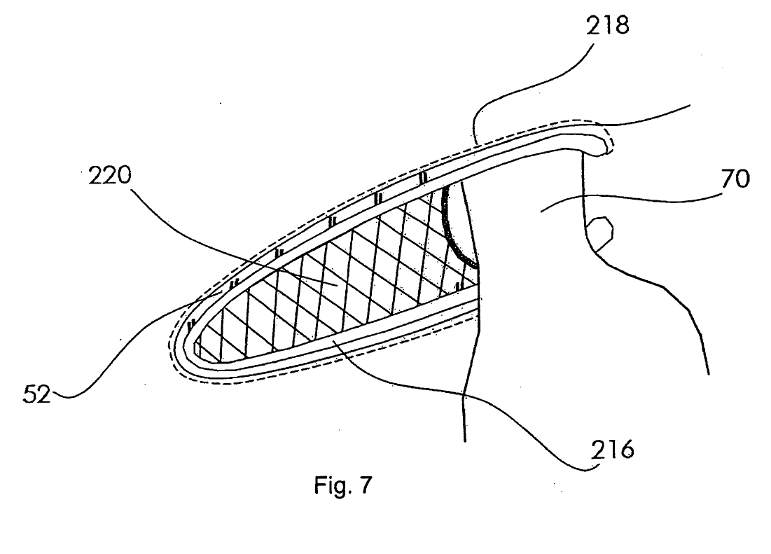

Now referring to FIGS. 6 and 7, FIGS. 6 and 7 depict diagrammatic, bottom views of one embodiment of the occlusion device 200 implanted for use to treat and/or prevent a hiatal hernia. In this embodiment, the occlusion device 200 is employed as a “Tension-Free” device because it avoids mechanical mesh fixation. By avoiding mechanical mesh fixation, the occlusion device 200 prevents injuries to the vital structure surrounding the esophageal hiatus and provides for ease in insertion and removal from a patient. Additionally, employing a soft biologic or synthetic mesh avoids the formation of a visceral adhesion, local fibrosis (dysphagia), or esophageal erosion.

In FIG. 6, the occlusion device 200 is shown coupled with the diaphragmatic wall 52 and the esophagus 70 in a location adjacent to the stomach 60. In this embodiment, the first and second magnetic components 212, 214 each comprise a C-shaped configuration and have an open end and a closed end. In addition, the opening 221 of the second magnetic component 214 comprises a diameter that closely approximates the diameter of the esophagus 70 at the EG junction.

In application, the first magnetic component 212 is positioned adjacent to the inferior wall of the diaphragm 52 with the open end of the first magnetic component 212 positioned around the esophagus 70. The second magnetic component 214 is positioned adjacent to the superior wall of the diaphragm 52 such that the second magnetic component 214 is positioned around the esophagus 70. Due to the size of the opening 221, only the esophagus 70 is able to fit therethrough. The occlusion device 200 may be applied to the diaphragm 52 in a posterior or anterior placement, depending on the site of the herniation. While the occlusion device 200 is shown in the anterior placement in FIG. 6, FIG. 7 illustrates the occlusion device 200 as applied to the diaphragm 52 in the posterior placement.

Due to the close proximity of the first magnetic component 212 and the second magnetic component 214, the first and second magnetic components 212, 214 magnetically engage through the diaphragm wall 52, compressing the diaphragm wall 52 therebetween. In one embodiment, the mesh 220 is coupled with the inferior wall of the diaphragm 52 due to the magnetic forces exerted by the first and second magnetic components 212, 214. In this embodiment, because the mesh 220 is coupled with the diaphragm 52, after implantation, diaphragmatic tissue may grow into the mesh 220. This tissue growth further secures the occlusion device 200 to the diaphragm 52.

In the embodiments shown in FIGS. 6 and 7, the size of the opening 221 of the first magnetic component 212 is equivalent to the diameter of the esophagus 70; therefore the mesh 220 occludes any portion of an esophageal hiatus that the esophagus 70 does not occupy. By covering any unnecessary space of the esophageal hiatus and/or any weakened diaphragmatic tissue, the placement and structure of the occlusion device 200 prevent the stomach 60 from protruding through the diaphragm wall 52 and into the mediastinal cavity via the esophageal hiatus.

The structure and placement of the occlusion device 200 further reduces the risk that the occlusion device 200 will become displaced or dislodged over time. The magnetic force between the first magnetic component 212 and the second magnetic component 214, the diaphragmatic tissue growth into the mesh 220, and the first and second magnetic components 212, 214 both partially surrounding the esophagus 70 all assist in anchoring the occlusion device 200 in its desired position. In the embodiment wherein the occlusion device 200 further comprises a plurality of barbs 222, the plurality of barbs 222 further secure the occlusion device 200 to the diaphragm wall 52 and prevent the occlusion device 200 from shifting or becoming dislodged.

Depending on the configuration of the barbs 222, the barbs 222 may form a wave-like pattern in the diaphragm tissue 52 and increase the amount of force required to dislodge the occlusion device 200 from the diaphragm 52. In an additional embodiment, the barbs 222 are configured as sharp points such that the barbs 222 puncture the diaphragmatic tissue, thus functioning similarly to sutures or staples by securing the occlusion device 200 to the diaphragm 52. Even though the barbs 222 may puncture the diaphragmatic tissue, the barbs 222 will not produce the complications seen with the sutures and staples used in the prior art because the barbs 222 produce less ischemic effect in the diaphragmatic muscles. In these ways, the barbs 222 can provide further resistance and prevent the occlusion device 200 from shifting relative to the esophagus 70 or the diaphragm 52.

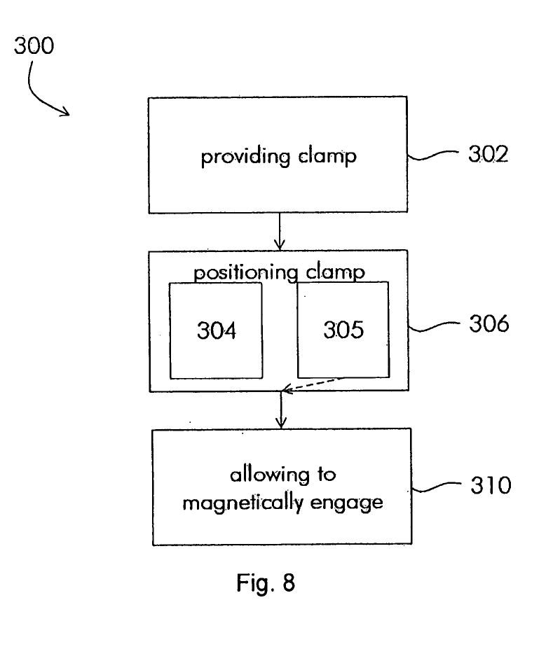

FIG. 8 shows a flow chart of one embodiment of a method 300 for occluding a tissue opening. For ease of understanding, the steps of the related methods described herein will be discussed relative to the components of the occlusion device 200 shown in FIGS. 5-7, but it will be appreciated by one skilled in the art that any such device can be used to perform these methods, so long as the device is capable of magnetically engaging a magnetic composition through a piece of tissue such that the engagement is secure.

Generally, a user can utilize the occlusion device 200 shown in FIGS. 5-7 to occlude a tissue opening, and specifically to treat and/or prevent a hiatal hernia. As shown in FIG. 8, step 302 comprises providing the occlusion device 200, or a similar device capable of occluding an opening in a tissue. At step 306, the occlusion device 200 is adhered to the diaphragm 52 in such a manner that at least a portion of the mesh 220 occludes a portion of the hiatus. In one embodiment of the method 300, step 306 may comprise two independent steps. Specifically, step 304 comprises positioning the first magnetic component 212 of the occlusion device 200 adjacent to the inferior portion of the diaphragm 52 (i.e. within the abdominal cavity) and around the esophagus 70. Step 305 comprises positioning the second magnetic component 214 adjacent to the superior portion of the diaphragm (i.e. within the chest cavity) and inserting the esophagus into the opening 221. Due to the bias between the first and second magnetic components 212, 214, aligning the two components 212, 214 with one another on opposite sides of the diaphragm 52 is an uncomplicated process, even when the occlusion device 200 is being implanted through laparoscopic technique. When the first and second magnetic components 212, 214 are substantially aligned, the mesh 220 effectively occludes the excess space in the esophageal hiatus, thus allowing only the esophagus 70 to traverse the diaphragm wall 52.

Once the components of the occlusion device 200 are positioned on opposite sides of the diaphragm 52 and properly aligned with the esophagus 70 and esophageal hiatus, at step 310 the first magnetic component 212 and the second magnetic component 214 are allowed to magnetically engage. Because of the placement of the components relative to the diaphragm 52 and the esophagus 70, the diaphragmatic tissue 52 is compressed between the first magnetic component 212 and the second magnetic component 214, and the esophagus 70 is restrained within the opening 221. The arrangement of the occlusion device 200 relative to the diaphragm 52 and the esophagus 70 anchors the occlusion device 200 to the diaphragm 52 and secures the mesh 220 such that the mesh 220 firmly blocks the hiatus. Due to the size, configuration, and simple implantation procedure of the occlusion device 200, the occlusion device 200 may be inserted into the body cavity laparoscopically, thereby reducing the number of incisions required and the amount of stress involved with administering treatment.

It will be appreciated that the devices and methods described herein provide several advantages over the devices and processes of the prior art. The magnetic devices 10, 200 can be inserted laparoscopically, which is a less invasive procedure and significantly decreases the patient's stress and recovery time. In addition, particularly with respect to the device 200, pressure is not exerted on the crura of the diaphragm as is common with procedures and devices of the prior art.

While the occlusion devices 10, 200 are presented with respect to the stomach and ulcer restriction anatomy, as one of ordinary skill in the art would recognize, the occlusion devices 10, 200 and the methods 200, 300 may be expanded to any organ, limb or body structure that would benefit from reshaping or remodeling using reversible, easy to use, and easy to implement techniques.

The devices and methods have been presented in detail with reference to certain embodiments thereof; however, such embodiments are offered by way of non-limiting examples, as other versions are possible. It is anticipated that a variety of other modifications and changes will be apparent to those having ordinary skill in the art and that such modifications and changes are intended to be encompassed within the spirit and scope of the devices and methods as defined by the following claims.

1. An apparatus for occluding a tissue opening, comprising:
at least one magnetic component, the at least one magnetic component for placement on a tissue and biased to decrease the size of an opening in the tissue.

2. The apparatus of claim 1, wherein the at least one magnetic component comprises a first magnetic component and a second magnetic component.

3. The apparatus of claim 1, wherein the at least one magnetic component is flexible.

4. The apparatus of claim 1, wherein the at least one magnetic component is semi-flexible.

5. The apparatus of claim 1, wherein the at least one magnetic component is articulated.

6. The apparatus of claim 1, wherein the at least one magnetic component comprises a first bar and a second bar biased toward each other when in proximity.

7. An apparatus for occluding a tissue opening, comprising:
a first magnetic component and a second magnetic component, such that when the first magnetic component and the second magnetic component are in proximity, the first magnetic component and the second magnetic component magnetically engage and hold tissue therebetween.

8. An apparatus for occluding a tissue opening, comprising:
a first component that comprises a magnetic shape defining an interior and a section of mesh disposed across a portion of the interior of the first component;
a second component having a magnetic shape that matches at least a portion of the magnetic shape of the first component;
wherein when the first component and the second component are in proximity to each other, the first component and the second component are biased toward each other to thereby compress any tissue disposed therebetween.

9. The apparatus of claim 8, wherein the mesh defines an opening, the opening configured to allow an esophagus to traverse therethrough.

10. The apparatus of claim 8, wherein the magnetic shape of the first component is selected from the group consisting of a U-shape, a C-shape, a rectangular shape, and a V-shape.

11. The apparatus of claim 8, wherein the mesh extends radially inward within the interior of the first component.

12. The apparatus of claim 8, wherein the second component further comprises an interior and a section of mesh disposed on a portion of the interior thereof.

13. The apparatus of claim 8, wherein the first component and the second component further comprise a plurality of barbs.

14. The apparatus of claim 13, wherein the first component and the second component further comprise a plurality of indentations, each indentation configured to receive at least one of the plurality of barbs.

15. The apparatus of claim 8, wherein the first component and the second component are flexible.

16. The apparatus of claim 8, wherein the first component and the second component are semi-flexible.

17. The apparatus of claim 8, wherein the first component and the second component are articulated.

18. The apparatus of claim 8, wherein the section of mesh comprises a biocompatible non-reabsorbable material.

19. The apparatus of claim 8, wherein the mesh comprises a synthetic non-reabsorbable material.

20. A method for occluding a tissue opening comprising the steps of:
providing an apparatus comprising a first magnetic component and a second magnetic component, such that when the first magnetic component and the second magnetic component are in proximity, the first magnetic component and the second magnetic component magnetically engage and hold tissue therebetween; and
adhering the apparatus to a tissue having an opening such that at least a portion of the apparatus occludes a portion of the opening of the tissue.

21. The method of claim 20, wherein the tissue comprises a diaphragm having a superior wall and an inferior wall, and the opening comprises a hiatus.

22. The method of claim 20, wherein the first magnetic component further comprises a magnetic shape defining an interior and a section of mesh disposed across a portion of the interior of the first component.

23. The method of claim 20, further comprising the step of positioning the apparatus such that the mesh is positioned adjacent to the inferior wall of the diaphragm.

24. The method of claim 20, further comprising the step of positioning the apparatus such that the mesh is positioned adjacent to the superior wall of the diaphragm.

25. A method for reducing the size of a tissue opening comprising the steps of:
providing a magnetic apparatus for placement on a tissue having an opening with a first circumference, the magnetic apparatus biased to decrease the size of the opening in the tissue; and
placing the magnetic apparatus around at least a portion of the tissue to result in the opening having a second circumference, wherein the second circumference is less than the first circumference.

26. The method of claim 25, wherein the tissue comprises a diaphragm and the opening in a tissue comprises a hiatus.

27. The method of claim 25, wherein the magnetic apparatus further comprises barbs to affect the bias of the magnetic apparatus.

28. A method for occluding a tissue opening comprising the steps of:
providing an apparatus comprising a first component having a magnetic shape defining an interior and a section of mesh disposed across a portion of the interior of the first component, and a second component having a magnetic shape that matches at least a portion of the magnetic shape of the first component; wherein when the first component and the second component are in proximity to each other, the first component and the second component are biased toward each other to thereby compress any tissue disposed therebetween;
positioning the apparatus such that a tissue having an opening is located between the first component and the second component; and
allowing the first component and the second component to magnetically engage one another, thereby compressing the tissue therebetween, at least partially occluding the opening, and anchoring the apparatus to the tissue.

29. The method of claim 28, wherein the step of positioning the apparatus further comprises the steps of:
placing the first component adjacent to an inferior portion of a tissue; and
placing the second component adjacent to a superior portion of the tissue.

30. The method of claim 29, further comprising the steps of:
placing the first component around a portion of a protrusion projecting through the opening; and
placing the second component around a portion of the protrusion.

31. A method of treating and preventing a hiatal hernia comprising the steps of:
providing an apparatus having a first component having a magnetic shape defining an interior and a section of mesh disposed across a portion of the interior of the first component, and a second component having a magnetic shape that matches at least a portion of the magnetic shape of the first component; wherein when the first component and the second component are in proximity to each other, the first component and the second component are biased toward each other to thereby compress any tissue disposed therebetween;
placing the first component adjacent to an inferior portion of a diaphragm and around a thoracic portion of an esophagus;
placing the second component adjacent to a superior portion of the diaphragm and around an abdominal portion of an esophagus; and
allowing the first component and the second component to magnetically engage one another, thereby compressing the tissue therebetween, anchoring the apparatus to the tissue, and occluding the opening.

32. The method of claim 31, further comprising the step of adjusting the position of the mesh such that the mesh is adjacent to the esophagus.
